(19) United States
(12) Patent Application Publication
(10) Pub. No.: US 2026/0168976 A1
(43) Pub. Date: Jun. 18, 2026

(54) DETECTION SYSTEM AND DETECTION METHOD

(71) Applicant: PANASONIC HOUSING SOLUTIONS CO., LTD., Osaka (JP)

(72) Inventor: Hiroshi USHIO, Osaka (JP)

(21) Appl. No.: 19/127,331

(22) PCT Filed: Nov. 20, 2023
(86) PCT No.: PCT/JP2023/041671

§ 371 Date: May 5, 2025

(30) Foreign Application Priority Data
Nov. 25, 2022 (JP) 2022-188669

(51) Int. Cl. G01N 33/00

(52) U.S. Cl. CPC G01N 33/0047

(57) ABSTRACT

A detection system includes a plurality of detection units and a switching mechanism. Each of the plurality of detection units includes: a detector that detects at least one substance contained in a gas phase; a detecting path allowing a first gas, containing a substance vaporizing from a detection target, to flow therethrough; and a cleaning path allowing a second gas, not containing the substance vaporizing from the detection target, to flow therethrough. The switching mechanism switches an operating state of each of the plurality of detection units from one of a plurality of states to another. The plurality of states includes: a first state where the first gas is allowed to enter the detector through the detecting path; and a second state where the first gas is prohibited from entering the detector through the detecting path but the second gas is allowed to enter the detector through the cleaning path.

### TECHNICAL FIELD

The present disclosure generally relates to a detection system and a detection method, and more particularly relates to a detection system and detection method for detecting a substance vaporizing from a detection target.

### BACKGROUND ART

Patent Literature 1 discloses an odor measuring apparatus which performs: a providing step including providing an odor sensor for measuring the odor of a gas; a gas supplying step including supplying the gas to the odor sensor; and an odorless gas supplying step including supplying an odorless gas to the odor sensor for a predetermined cleaning time. The odor measuring apparatus performs the odorless gas supplying step compulsorily immediately after having performed the gas supplying step, thus causing the odor sensor to provide an output value representing the odor intensity of the gas to make the cleaning time correspond to the output value. According to the teaching of Patent Literature 1, this allows for providing an odor measuring apparatus and odor measuring method with a simple and handy configuration.

### CITATION LIST

### Patent Literature

Patent Literature 1: JP 2007-010326 A

### SUMMARY OF INVENTION

The problem to be overcome by the present disclosure is to provide a detection system and detection method which may not only reduce the chances of causing a decline in detection accuracy when detecting a substance vaporizing from a detection target but also increase the efficiency of detection when sequentially detecting the substance vaporizing from a plurality of detection targets.

A detection system according to an aspect of the present disclosure includes a plurality of detection units and a switching mechanism. Each of the plurality of detection units includes: a detector that detects at least one substance contained in a gas phase; a detecting path that allows a first gas, containing a substance vaporizing from a detection target, to flow therethrough; and a cleaning path that allows a second gas, not containing the substance vaporizing from the detection target, to flow therethrough. The switching mechanism switches an operating state of each of the plurality of detection units from one of a plurality of states to another. The plurality of states includes: a first state where the first gas is allowed to enter the detector through the detecting path; and a second state where the first gas is prohibited from entering the detector through the detecting path, but the second gas is allowed to enter the detector through the cleaning path.

A detection method according to another aspect of the present disclosure is a method for detecting, using a detection system, a substance vaporizing from a detection target. The detection system includes a plurality of detection units. Each of the plurality of detection units includes: a detector that detects at least one substance contained in a gas phase; a detecting path that allows a first gas, containing a substance vaporizing from a detection target, to flow therethrough; and a cleaning path that allows a second gas, not containing the substance vaporizing from the detection target, to flow therethrough. The detection method includes switching an operating state of each of the plurality of detection units from one of a plurality of states to another. The plurality of states includes: a first state where the first gas is allowed to enter the detector through the detecting path; and a second state where the first gas is prohibited from entering the detector through the detecting path, but the second gas is allowed to enter the detector through the cleaning path.

### BRIEF DESCRIPTION OF DRAWINGS

FIG. 1 is a schematic representation illustrating a first embodiment of the present disclosure;

FIG. 2 illustrates a cross-sectional view of a detector according to the first embodiment and a block diagram of a control module;

FIG. 3 is a schematic representation of a gas sensor included in the detector;

FIG. 4 is a graph showing, as an exemplary model, how the output of one detector may change with time;

FIG. 5 is a graph showing, as an exemplary model, how the respective outputs of a plurality of detectors may change with time; and

FIG. 6 is a schematic representation illustrating a second embodiment of the present disclosure.

### DESCRIPTION OF EMBODIMENTS

In a situation where a substance vaporizing from a detection target is supplied to, and detected by, a detector, attempting to sequentially and consecutively detect respective substances vaporizing from a plurality of detection targets will make it difficult to detect the substances accurately. This is because the respective substances vaporizing from the plurality of detection targets will mix with each other in the detector. In addition, particularly if the detector includes a sensor element, of which a physical property value such as an electrical resistance value is allowed to change when the detector adsorbs the substance, the substance cannot be detected accurately unless the substance adsorbed into the sensor element is desorbed from the sensor element. That is why the detector needs to be cleaned every time the detector has detected any substance vaporizing from the detection target (see, for example, Patent Literature 1 (JP 2007-010326 A)).

Nevertheless, no substance can be detected while the detector is being cleaned. Thus, attempting to sequentially and consecutively detect respective substances vaporizing from a plurality of detection targets would cause a decline in the efficiency of detection.

To overcome such a problem with the related art, the present disclosure provides a detection system which may not only reduce the chances of causing a decline in detection accuracy when detecting a substance vaporizing from a detection target but also increase the efficiency of detection when sequentially detecting respective substances vaporizing from a plurality of detection targets.

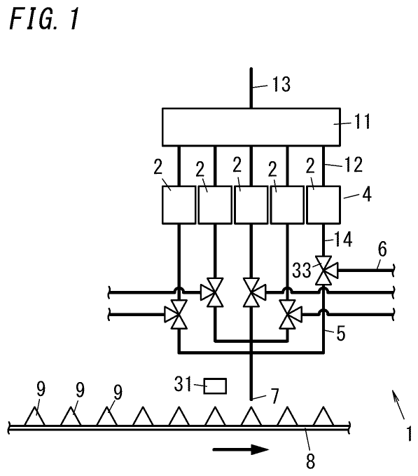

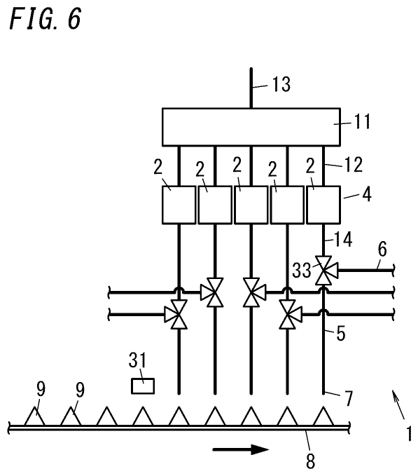

Embodiments and their variations will now be described with reference to FIGS. 1-6. Note that the embodiments and their variations to be described below are only exemplary ones of various embodiments of the present disclosure and their variations and should not be construed as limiting. Rather, the exemplary embodiments and their variations may be readily modified in various manners depending on a design choice or any other factor without departing from the scope of the present disclosure. Optionally, the configurations to be described later for the variations may be adopted in combination as appropriate. The drawings to be referred to in the following description of embodiments are all schematic representations. Thus, the ratio of the dimensions (including thicknesses) of respective constituent elements illustrated on the drawings does not always reflect their actual dimensional ratio.

A detection system 1 according to an exemplary embodiment includes a plurality of detection units 2 and a switching mechanism 3. Each of the plurality of detection units 2 includes: a detector 4 for detecting at least one substance contained in a gas phase; a detecting path 5 for allowing a first gas, containing a substance vaporizing from a detection target 9 (hereinafter referred to as a “detected gas”), to flow therethrough; and a cleaning path 6 for allowing a second gas, not containing the substance vaporizing from the detection target 9 (hereinafter referred to as a “cleaning gas”), to flow therethrough. The switching mechanism 3 switches an operating state of each of the plurality of detection units 2 from one of a plurality of states to another. The plurality of states includes: a first state where the detected gas is allowed to enter the detector 4 through the detecting path 5; and a second state where the detected gas is prohibited from entering the detector 4 through the detecting path 5 but the cleaning gas is allowed to enter the detector 4 through the cleaning path 6.

The substance may be detected by using this detection system 1 while switching the operating state of each of the plurality of detection units 2 between a plurality of states including the first state and the second state.

This embodiment allows each of the plurality of detection units 2 to detect the substance vaporizing from the detection target 9 in the first state and to clean the detector 4 in the second state. This reduces, in detecting the substance vaporizing from the detection target 9, the chances of causing a decline in detection accuracy even when sequentially detecting respective substances vaporizing from a plurality of detection targets 9.

A first embodiment and a second embodiment, which are included in various embodiments of the present disclosure, will now be described.

A detection system 1 according to the first embodiment includes a single inlet port 7. The beginning of the detecting path 5 of each of the plurality of detection units 2 communicates with the inlet port 7.

The detection system 1 further includes a placing mechanism 8. The placing mechanism 8 sequentially places the plurality of detection targets 9 one by one at a position where the detection target 9 faces the inlet port 7. The placing mechanism 8 includes a mechanism for sequentially moving the plurality of detection targets 9 along a path that passes through the position where each of the plurality of detection targets 9 faces the inlet port 7.

The switching mechanism 3 sequentially switches the respective operating states of the plurality of detection units 2 to the first state one after another by shifting the timing to switch the operating state of one of the plurality of detection units 2 from the timing to switch the operating state of another one of the plurality of detection units 2. Furthermore, the switching mechanism 3 synchronizes switching of the operating state with the placing mechanism's 8 placement of the detection targets 9. The switching mechanism 3 includes: a sensor 31 for sensing the presence or absence of any of the plurality of detection targets 9 at a particular position on the path along which the plurality of detection targets 9 move; and a control unit 32 for controlling, in accordance with a result of sensing by the sensor 31, the timing to switch the operating state. The switching mechanism 3 further includes three-way valves 33.

The first embodiment will be described in further detail.

The detection system 1 includes five detection units 2. Each of the five detection units 2 includes the detector 4, the detecting path 5, and the cleaning path 6. Each of the five detection units 2 further includes a delivery path 12 and an introductory path 14. Each of the detecting path 5, the cleaning path 6, the delivery path 12, and the introductory path 14 may be configured as a pipe, for example.

Any type of detector 4 may be used without limitation as long as the detector 4 outputs a result of detection indicating depending on at least one type of substance in the gas phase. The result of detection may have any form as long as the result depends on the substance. For example, the result of detection may be a numerical value or a pattern such as a waveform.

The detector 4 may include a gas sensor 20, for example. In that case, the result of detection may be, for example, either a signal output by the gas sensor 20 when at least one type of substance in the gas phase is supplied to the gas sensor 20 or a piece of information obtained by transforming the signal.

If the detector 4 includes the gas sensor 20, then the gas sensor 20 may include, for example, a sensor element Ax having an electrical resistance value that changes when adsorbing a substance. In that case, the gas sensor 20 may be a sensor array including a plurality of sensor elements Ax having mutually different sensitivities. In that case, the result of detection may be, for example, either a set of signals output by the plurality of sensor elements Ax or a set of pieces of information obtained by transforming the output signals. As can be seen, if the gas sensor 20 is a sensor array, various types of evaluation may be made based on the result of detection by reference to a combination of multiple pieces of information.

As used herein, the expression “the plurality of sensor elements Ax have mutually different sensitivities” means that the plurality of sensor elements Ax may detect mutually different substances and/or have mutually different detection sensitivities with respect to a given substance. Also, each of the plurality of sensor elements Ax may be sensitive to either only one type of substance or two or more types of substances, whichever is appropriate.

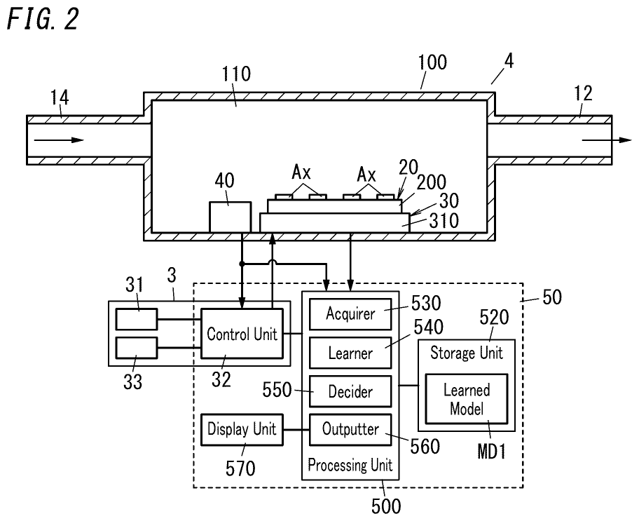

A specific example of the detector 4 is shown in FIG. 2. This detector 4 includes a sensor housing chamber 100, the gas sensor 20, a temperature control element 30, and a temperature sensor 40.

The gas sensor 20, the temperature control element 30, and the temperature sensor 40 are housed in a housing space 110 inside the sensor housing chamber 100. The delivery path 12 and the introductory path 14 are connected to the sensor housing chamber 100.

The temperature control element 30 is an element for heating the gas sensor 20, and may be, for example, an electro-thermal element 310 that generates heat when energized. The gas sensor 20 is disposed over the electro-thermal element 310. The temperature sensor 40 may be, for example, a thermistor and is disposed in the vicinity of the gas sensor 20 inside the housing space 110. The temperature sensor 40 is a sensor for detecting the temperature of the gas sensor 20 and may detect the temperature of the gas sensor 20 indirectly by detecting the temperature of the space surrounding the gas sensor 20 (i.e., the temperature of the housing space), for example. The temperature control element 30 is controlled by the control unit 32 as will be described later.

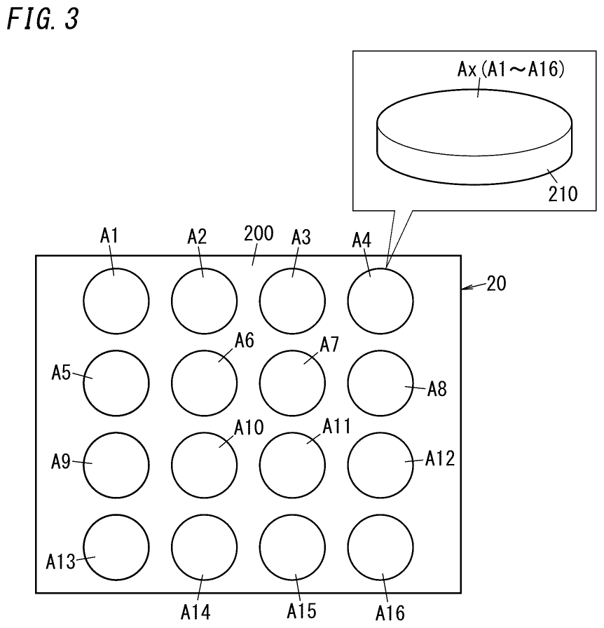

The gas sensor 20 is a sensor array including a plurality of sensor elements Ax having mutually different sensitivities. In this example, the gas sensor 20 includes sixteen sensor elements Ax, which will be hereinafter sometimes referred to as “sensor elements A1-A16” (refer to FIG. 3). The sixteen sensor elements A1-A16 are arranged in four rows and four columns on a board 200. Note that the number of the sensor elements Ax may be changed as appropriate. Also, the plurality of sensor elements Ax may be arranged in any pattern without limitation. Alternatively, the plurality of sensitive elements may be arranged in line. Still alternatively, the plurality of sensitive elements may also be arranged at intervals to form a single circular pattern or a pattern consisting of plurality of concentric circles.

Each of the plurality of sensor elements Ax may include, for example, a matrix containing an organic material and electrically conductive particles dispersed in the matrix. Each of the sensor elements Ax shown in FIG. 3 has the shape of a circular membrane when viewed in plan. However, this is only an exemplary shape of each sensor element Ax and should not be construed as limiting.

As the organic material, a material having the property of adsorbing at least one type of substance in the gas phase may be selected. The organic material contains, for example, at least one selected from the group consisting of chromatographic column fillers OV-17, OV-22, OV-25, OV-225, OV-330, SILAR-5CP, SILAR-7CP, and OV-275 manufactured by Shinwa Chemical Industries, Ltd., and polystyrene, poly(4-tert-butylstyrene), poly(isobutyl methacrylate), poly(butyl methacrylate), polyvinyl formal, poly(ethylene succinate), low-molecular-weight poly(vinylidene fluoride), and high-molecular-weight poly(vinylidene fluoride).

If the gas sensor 20 includes a plurality of sensor elements Ax containing mutually different organic materials, then the plurality of sensor elements Ax may have mutually different sensitivities. Note that the organic materials enumerated above are only examples and should not be construed as limiting.

The electrically conductive particles include at least one material selected from the group consisting of, for example, carbon materials, electrically conductive polymers, metals, metal oxides, semiconductors, superconductors, and complex compounds.

As the organic material contained in each sensor element Ax adsorbs the substance, the matrix comes to have an increased volume, thus causing an increase in the distance between the electrically conductive particles in the sensor element Ax. As a result, the electrical resistance value of the sensor element Ax increases accordingly. The larger the amount of a marker component adsorbed into the organic material is, the higher the electrical resistance value of each sensor element Ax is. Thus, the change in the electrical resistance value of each sensor element Ax is a piece of information dependent on the amount of the substance in the gas phase.

The board 200 includes an electrode connected to each sensor element Ax. Upon the application of a voltage from the electrode to the sensor element Ax, an electric current flows through the sensor element Ax in an amount corresponding to its own electrical resistance value. Either an electric current corresponding to the electrical resistance value or information obtained by transforming the electric current is acquired as the output of each sensor element Ax. A set of the respective outputs of the sensor elements Ax is the result of detection obtained by the sensor device.

The detection system 1 further includes a control module 50 as shown in FIG. 2. The control module 50 includes a processing unit 500, a control unit 32, a storage unit 520, and a display unit 570.

The processing unit 500 is a control circuit for controlling the operation of the detector 4. The control unit 32 is a control circuit which belongs to the switching mechanism 3 and controls the operation of the switching mechanism 3.

The processing unit 500 includes not only a decider 550 but also an acquirer 530, a learner 540, and an outputter 560 as shown in FIG. 2. In FIG. 2, the acquirer 530, the learner 540, the decider 550, and the outputter 560 do not have a substantive physical configuration but just represent respective functions to be performed by the processing unit 500.

The acquirer 530 acquires the result of detection provided by the detector 4.

The learner 540 makes an artificial intelligence program (algorithm) machine-learn training data, thereby generating a learned model. The learner 540 has the learned model stored in the storage unit 520. That is to say, the learner 540 has the combination of the result of detection obtained by the detector 4 and decision results stored as training data in the storage unit 520 and is in charge of a learning phase of generating a learned model MD1 based on the training data. Optionally, the learner 540 may attempt to improve the performance of the learned model MD1 by making re-learning using training data that the acquirer 530 has newly collected after the learned model MD1 has been generated.

The decider 550 makes various types of decisions based on the result of detection by using the learned model MD1 stored in the storage unit 520.

The outputter 560 outputs the results of decisions made by the decider 550 to the display unit 570.

The storage unit 520 includes one or more storage devices. Examples of the storage devices include a RAM, a ROM, and an EEPROM. The storage unit 520 stores the learned model MD1, for example. The learned model MD1 may be generated by a learning phase using the detector 4 as described above. Alternatively, the learned model MD1 may also be generated by a learning system other than the detector 4. If the learned model MD1 is generated by a learning system other than the detector 4, then the detector 4 does not have to include the learner 540.

The display unit 570 externally displays, in a form recognizable for human beings, the result of decision provided by the outputter 560. The display unit 570 may be, for example, a device for displaying the result of decision in a visual form. In that case, the display unit 570 includes a display device such as a liquid crystal display. Alternatively, the display unit 570 may also be a device for outputting the result of the decision as a sound or a voice. In that case, the display unit 570 may include either a buzzer or a loudspeaker, for example.

The specifics of the result of the decision are not limited to any particular ones. For example, the result of the decision may indicate the state, quality, or any other parameter of the detection target 9 depending on, for example, the type of the detection target 9.

Each of the processing unit 500 and the control unit 32 may be implemented as, for example, a computer system including one or more processors (microprocessors) and one or more memories. The computer system performs the functions of the processing unit 500 or the control unit 32 by making the one or more processors execute one or more programs (applications) stored in the one or more memories. In this embodiment, the program is stored in advance in either the memory of each of the processing unit 500 and the control unit 32 or the storage unit 520. Alternatively, the program may also be downloaded through a telecommunications line such as the Internet or be distributed after having been recorded in some non-transitory storage medium such as a memory card. Specifically, each of the processing unit 500 and the control unit 32 includes a computer system. The computer system includes a processor and a memory as principal hardware components thereof. The computer system performs the functions of the detection system 1 according to the present disclosure by making the processor execute a program stored in the memory of the computer system. The program may be stored in advance in the memory (such as the storage unit 520) of the computer system. Alternatively, the program may also be downloaded through a telecommunications line or be distributed after having been recorded in some non-transitory storage medium such as a memory card, an optical disc, or a hard disk drive, any of which is readable for the computer system. The processor of the computer system may be made up of a single or a plurality of electronic circuits including a semiconductor integrated circuit (IC) or a large-scale integrated circuit (LSI). As used herein, the “integrated circuit” such as an IC or an LSI is called by a different name depending on the degree of integration thereof. Examples of the integrated circuits such as an IC or an LSI include integrated circuits called a “system LSI,” a “very-large-scale integrated circuit (VLSI),” and an “ultra-large-scale integrated circuit (ULSI).” Optionally, a field-programmable gate array (FPGA) to be programmed after an LSI has been fabricated or a reconfigurable logic device allowing the connections or circuit sections inside of an LSI to be reconfigured may also be adopted as the processor. Those electronic circuits may be either integrated together on a single chip or distributed on multiple chips, whichever is appropriate. Those multiple chips may be aggregated together in a single device or distributed in multiple devices without limitation. As used herein, the “computer system” includes a microcontroller including one or more processors and one or more memories. Thus, the microcontroller may also be implemented as a single or a plurality of electronic circuits including a semiconductor integrated circuit or a large-scale integrated circuit.

In the embodiment described above, the plurality of functions of the control module 50 are aggregated together in a single housing. However, this is not an essential configuration for the detection system 1. Alternatively, those constituent elements of the control module 50 may be distributed in multiple different housings. Optionally, at least some functions of the detector 4 may be implemented as, for example, a cloud computing system as well.

The beginning of the delivery path 12 and the end of the introductory path 14 are connected to the sensor housing chamber 100 of the detector 4. This allows a gas flowing through the introductory path 14 to be supplied onto the detection unit 2 and also allows a gas inside the detection unit 2 to be delivered through the delivery path 12. The end of the detecting path 5 and the end of the cleaning path 6 are connected to the beginning of the introductory path 14.

The beginning of the cleaning path 6 communicates with a cleaning gas supply source. Any cleaning gas may be used without limitation as long as the cleaning gas may be used to clean the detector 4. The cleaning gas may be, for example, clean air. The cleaning gas supply source may be, for example, the outside air or a gas cylinder.

The respective ends of the five detecting paths 5 are confluent with each other and connected to the single inlet port 7.

The detection system 1 further includes a pump 11 and an exhaust path 13 connected to the pump 11. The respective ends of the delivery paths 12 of the five detection units 2 are all connected to the pump 11. The pump 11 may operate to pump out the gas inside the detectors 4 through the delivery paths 12 and exhaust the gas through the exhaust path 13.

The placing mechanism 8 according to the first embodiment is implemented as a conveyor belt for moving the plurality of detection targets 9 relatively with respect to the inlet port 7 by carrying the plurality of detection targets 9 continuously. The conveyor belt passes under the inlet port 7. This allows the placing mechanism 8 to sequentially move the plurality of detection targets 9 along a path that passes through a position where each of the detection targets 9 faces the inlet port 7.

The switching mechanism 3 includes the sensor 31, the control unit 32, and the three-way valves 33 as described above.

Each of the three-way valves 33 is provided for a corresponding one of the five detection units 2. Each three-way valve 33 is provided at a point where the end of its corresponding detecting path 5, the end of its corresponding cleaning path 6, and the beginning of its corresponding introductory path 14 are confluent with each other. That is to say, the end of the detecting path 5 and the end of the cleaning path 6 are connected to the beginning of the introductory path 14 via the three-way valve 33. The three-way valve 33 may switch between a state where the end of the detecting path 5 communicates with the beginning of the introductory path 14 but the end of the cleaning path 6 does not communicate with the beginning of the introductory path 14 (hereinafter referred to as a “detecting state”) and a state where the end of the detecting path 5 does not communicate with the beginning of the introductory path 14 but the end of the cleaning path 6 communicates with the beginning of the introductory path 14 (hereinafter referred to as a “cleaning state”). Note that the detecting state of the three-way valve 33 corresponds to the first state of the detection unit 2 and the cleaning state of the three-way valve 33 corresponds to the second state of the detection unit 2. That is to say, according to the first embodiment, in the first state, the detected gas is allowed to enter the detector 4 through the detecting path 5 and the cleaning gas is prohibited from entering the detector 4 through the cleaning path 6. In the second state, on the other hand, the detected gas is prohibited from entering the detector 4 through the detecting path 5 and the cleaning gas is allowed to enter the detector 4 through the cleaning path 6.

The sensor 31 may be an optical sensor such as a photoelectric sensor, a fiber sensor, a laser sensor, or an image sensor. The sensor 31 senses the presence or absence of any detection target 9 at a particular position on the path along which the detection target 9 is caused to move by the placing mechanism 8. The particular position is located opposite, in the direction in which the detection target 9 moves, from the position where the detection target 9 faces the inlet port 7. This allows the timing at which the detection target 9 is going to be placed at the position where the detection target 9 faces the inlet port 7 to be checked based on the result of sensing of the detection target 9 by the sensor 31.

The control unit 32 is a control circuit designed to control the switching mechanism 3 which is included in the control module 50 as described above.

The control unit 32 receives the result of sensing obtained by the sensor 31 and controls the three-way valves 33 in accordance with the result of sensing.

Specifically, first, the control unit 32 controls the respective three-way valves 33 such that the three-way valve 33 of each of the plurality of detection units 2 is in the cleaning state. That is to say, the switching mechanism 3 turns each of the plurality of detection units 2 into the second state.

Next, after the sensor 31 has sensed any detection target 9 at a point in time, the control unit 32 will switch the three-way valve 33 in one of the plurality of detection units 2 to the detecting state when a certain amount of time passes since the point in time. After that, when another certain amount of time passes since then, the control unit 32 will switch the three-way valve 33 to the cleaning state. That is to say, the switching mechanism 3 switches the operating state of one of the plurality of detection units 2 from the second state to the first state when the certain amount of time passes since the sensor 31 has sensed the detection target 9 and then switches the operating state of the detection unit 2 from the first state to the second state when the certain amount of time further passes after that. This timing to switch the operating state is set such that the detection target 9 sensed by the sensor 31 during the interval between a point in time when the operating state of the detection unit 2 has been switched to the first state and a point in time when its operating state is switched to the second state is placed by the placing mechanism 8 at the position where the detection unit 2 faces the inlet port 7.

Next, the control unit 32 switches, when a certain amount of time has passed since the point in time when the sensor 31 sensed another detection target 9, the operating state of the three-way valve 33 of another detection unit 2, different from the previous one, in the same way. That is to say, the switching mechanism 3 switches, in the same way, the operating state of another detection unit 2 different from the detection unit 2, of which the operating state has been switched last time.

By repeating this operation, the switching mechanism 3 sequentially switches the respective operating states of the plurality of detection units 2 one after another every time the sensor 31 senses any detection target 9. In this manner, the switching mechanism 3 makes the plurality of detection units 2 sequentially perform a series of operating state switches such that their operating state switches from the second state to the first state and then switches to the second state again.

Also, in the second state, the control unit 32 heats the gas sensor 20 for a certain amount of time by causing a current to flow through the electro-thermal element 310. A temperature control unit 510 controls the temperature control element 30 based on the result of detection by the temperature sensor 40 to cause the temperature of the gas sensor 20 to increase to a predetermined temperature when the gas sensor 20 is heated. The predetermined temperature varies depending on the type of an organic material or any other constituent material included in each sensor element Ax but may be, for example, equal to or higher than 60° C. and equal to or lower than 110° C. Meanwhile, the duration for which the gas sensor 20 is heated may be equal to or shorter than a period of time for which the detection unit 2 stays in the second state. As the temperature of the gas sensor 20 increases due to heating, desorption of any substance adsorbed to the sensor element Ax is accelerated.

Next, it will be described how the detection system 1 according to the first embodiment operates.

According to the first embodiment, when the detection system 1 starts operating, the pump 11 and the placing mechanism 8 are activated. When the detection system 1 starts operating, the operating state of each of the plurality of detection units 2 is the second state. In this situation, the switching mechanism 3 sequentially switches the respective operating states of the plurality of detection units 2 and the processing unit 500 processes the result of detection provided by the detector 4 of each of the plurality of detection units 2. This allows the processing unit 500 to acquire the result of detection based on the substance vaporizing from the detection target 9 or to further make a decision, for example, based on the result of detection.

Next, it will be described how each of the plurality of detection units 2 operates.

As described above, when the detection system 1 starts operating, the operating state of each detection unit 2 is the second state. In the second state, a gas is caused by the pump 11 to flow through a gas flow channel of the detection unit 2. The three-way valve 33 is being cleaned in the second state. Thus, in the second state, the cleaning gas flows through the cleaning path 6 and the introductory path 14 in this order to enter the sensor housing chamber 100 of the detector 4 while the detected gas is prohibited from entering the detector 4 through the detecting path 5.

The sensor 31 senses that one detection target 9 is currently located at a particular position on the path, along which the detection targets 9 are caused to move one after another by the placing mechanism 8, and forwards the result of sensing to the control unit 32. When a certain amount of time passes since the point in time when the sensor 31 made the sensing, the control unit 32 switches the three-way valve 33 from the cleaning state to the detecting state. In this manner, the switching mechanism 3 switches the operating state of the detection unit 2 from the second state to the first state.

Even in the first state, a gas is also caused by the pump 11 to flow through the gas flow channel of the detection unit 2. In the first state, when the detection target 9 is placed by the placing mechanism 8 at a position where the detection target 9 faces the inlet port 7, a detected gas, including the substance vaporizing from the detection target 9, flows through the inlet port 7 into the detecting path 5. The detected gas flows through the detecting path 5 and the introductory path 14 in this order to enter the sensor housing chamber 100 of the detector 4, while the gas (i.e., cleaning gas) is prohibited from entering the detector 4 through the cleaning path 6.

In the detector 4, the sensor element Ax is exposed to the detected gas. The substance, vaporizing from the detection target 9, of the detected gas is adsorbed onto the sensor element Ax. As a result, the output signal of the sensor element Ax comes to have an increased value. The detector 4 outputs information about the output signal of the sensor element Ax as the result of detection.

Subsequently, when a certain amount of time passes since the point in time when the operating state of the detection unit 2 switched to the first state, the control unit 32 switches the three-way valve 33 from the detecting state to the cleaning state. In this manner, the switching mechanism 3 switches the operating state of the detection unit 2 from the first state to the second state. Meanwhile, while the operating state of the detection unit 2 is the second state, the control unit 32 causes a current to flow through the electro-thermal element 310 based on the result of detection by the temperature sensor 40, thereby heating the gas sensor 20 for a certain amount of time. This causes the cleaning gas to flow through the cleaning path 6 and the introductory path 14 in this order to enter the sensor housing chamber 100 of the detector 4, while the gas is prohibited from entering the detector 4 through the detecting path 5. As a result, the detected gas, which has surrounded the gas sensor 20 inside the sensor housing chamber 100 of the detector 4, is pushed away by the cleaning gas to flow out of the sensor housing chamber 100 into the delivery path 12 and then exhausted through the exhaust path 13. In addition, the substance is desorbed from the sensor element Ax of the gas sensor 20 and is also exhausted. Heating the gas sensor 20 accelerates the desorption of the substance from the sensor element Ax. This causes a decrease in the value of the output signal of the sensor element Ax. As a result, the detector 4 is cleaned, thus allowing the substance vaporizing from the detection target 9 to be detected accurately next time.

Furthermore, in the second state, the gas is prohibited from entering the detector 4 through the detecting path 5. This reduces, even if one detection target 9 is placed at a position where the detection target 9 faces the inlet port 7 and then another detection target 9 is placed at the position where the detection target 9 faces the inlet port 7, the chances of the substance vaporizing from the latter detection target 9 entering the detector 4. Consequently, the result of detection is less likely to be affected by the substance vaporizing from the latter detection target 9, thus enabling accurate detection of the detection target 9.

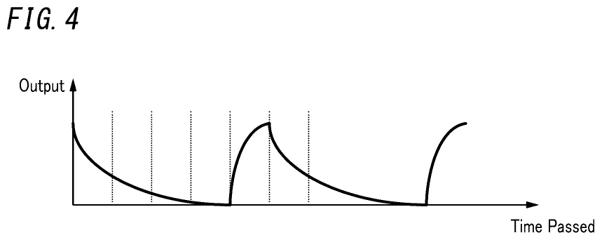

FIG. 4 shows, as a model, how the output of the detector 4 of one detection unit 2 changes with the passage of time in a situation where the detection unit 2 has operated as described above. In the first embodiment, the result of detection provided by the detector 4 is actually either the output signals of a plurality of sensor elements Ax or a set of pieces of information obtained by transforming the output signals. In FIG. 4 on the other hand, the degree of variation in a physical property value, sensitive to a substance, of all of the plurality of sensor elements Ax is regarded as the output of the detector 4 for the sake of convenience. As the detection unit 2 is controlled as described above to have its operating state switched alternately from the first state to the second state, or vice versa, the detected gas is supplied in the first state to the detector 4 to cause an increase in the output and then the cleaning gas is supplied in the second state to the detector 4 to clean the detector 4 and cause a decrease in the output as shown in FIG. 4. If the detection unit 2 operates in this way, then the substance may be detected accurately by repeating detection of the substance by the detection unit 2 and cleaning of the detection unit 2 a number of times. In addition, in the second state, the gas is prohibited from entering the detector 4 through the detecting path 5 as described above. Thus, the output of the detector 4 decreases without being affected by the substance vaporizing from any other detection target 9, thus enabling accurate detection of the substance as well. Nevertheless, in the second state, the detection unit 2 cannot detect any substance. That is to say, there arises a period in which the detection unit 2 cannot detect any substance.

In contrast, according to the first embodiment, the detection system 1 operates as described above to allow the switching mechanism 3 to sequentially switch the respective operating states of the plurality of detection units 2 one after another by shifting the timing to activate one of the plurality of detection units 2 from the timing to activate another detection unit 2. At this time, the placing mechanism 8 sequentially places one of the plurality of detection targets 9 after another at the position where the detection target 9 faces the inlet port 7 and the switching mechanism 3 synchronizes switching of the operating state of the detection unit 2 with the placing mechanism's 8 placement of the detection target 9. Thus, while one detection unit 2 is in the second state and the detector 4 of that detection unit 2 is being cleaned, the other detection units 2 sequentially detect the substance.

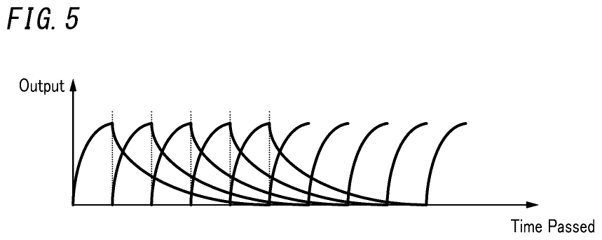

FIG. 5 shows, as a model, how the respective outputs of the detectors 4 in a plurality of detection units 2 change with the passage of time. In FIG. 5, the respective curves, each representing such relationship, are superposed one on top of another. In FIG. 5, the degree of variation in a physical property value, sensitive to a substance, of all of the plurality of sensor elements Ax is also regarded as the output of the detector 4 for the sake of convenience. According to the first embodiment, the plurality of detection units 2 may sequentially detect the substances as shown in FIG. 5. Thus, even though there arises a period during which each detection unit 2 cannot detect the substance while the substances vaporizing from the plurality of detection targets 9 are sequentially detected, the detection may be made as described above by sequentially using the plurality of detection units 2, thus allowing the efficiency of detection to be increased.

The result of detection provided by the detector 4 may be acquired by the processing unit 500 as described above. Alternatively, a decision may also be made based on the result of detection, for example. In that case, the result of detection may include not only the output of the detector 4 that has increased in the first state but also the output of the detector 4 that has decreased in the second state. As described above, according to the first embodiment, the output of the detector 4 decreases in the second state without being affected by the substance vaporizing from another detection target 9, and therefore, it heavily depends on the substance vaporizing from the detection target 9 how the output decreases. That is why if the result of detection includes the decrease in the output in the second state, then the result of detection may be used to make a wider variety of decisions, for example.

Next, a second embodiment will be described. In the following description, any constituent element of the detection system 1 according to this second embodiment, having the same function as a counterpart of the detection system 1 according to the first embodiment described above, will be designated by the same reference numeral as that counterpart's, and description thereof will be omitted as appropriate herein.

In the second embodiment, the detection system 1 includes a plurality of inlet ports 7. The number of the inlet ports 7 provided is the same as the number of the detection units 2 provided. The beginning of each of the detecting paths 5 of the plurality of detection units 2 communicates with a corresponding one of the plurality of inlet ports 7.

The detection system 1 further includes a placing mechanism 8. The placing mechanism 8 sequentially places the plurality of detection targets 9 one by one at a plurality of positions, at each of which one of the plurality of detection targets 9 faces a corresponding one of the plurality of inlet ports 7. The placing mechanism 8 includes a mechanism that sequentially moves the plurality of detection targets 9 along a path that sequentially passes through the positions where the plurality of detection targets 9 face the plurality of inlet ports 7.

The switching mechanism 3 switches the respective operating states of the plurality of detection units 2 to the first state simultaneously. Furthermore, the switching mechanism 3 synchronizes switching of the operating states with the placing mechanism's 8 placement of the detection targets 9. The switching mechanism 3 includes: a sensor 31 that senses the presence or absence of any of the plurality of detection targets 9 at a particular position on the path along which the plurality of detection targets 9 move; and a control unit 32 that controls, in accordance with a result of sensing by the sensor 31, the timing to switch the operating state. The switching mechanism 3 further includes three-way valves 33.

The second embodiment will now be described in further detail.

The detection system 1 includes five detection units 2. Each of the five detection units 2 includes the detector 4, the detecting path 5, and the cleaning path 6. Each of the five detection units 2 further includes the delivery path 12 and the introductory path 14.

The detection system 1 further includes the control module 50. The control module 50 includes the processing unit 500, the control unit 32, the storage unit 520, and the display unit 570.

The detection system 1 includes five inlet ports 7 which are as many as the detection units 2. The ends of the five detecting paths 5 are connected to the five inlet ports 7, respectively. The five inlet ports 7 are arranged side by side at intervals.

The detection system 1 further includes the pump 11 and the exhaust path 13 connected to the pump 11. The respective ends of the delivery paths 12 of the five detection units 2 are all connected to the pump 11. The pump 11 may operate to pump out the gas inside the detector 4 through the delivery path 12 and exhaust the gas through the exhaust path 13.

The placing mechanism 8 according to the second embodiment is implemented as a conveyor belt for moving the plurality of detection targets 9 relatively with respect to the inlet ports 7 by carrying the plurality of detection targets 9 continuously. The conveyor belt sequentially passes under the plurality of inlet ports 7. This allows the placing mechanism 8 to sequentially move the plurality of detection targets 9 along a path that sequentially passes through the plurality of positions where the detection targets 9 face the plurality of inlet ports 7.

The switching mechanism 3 includes the sensor 31, the control unit 32, and the three-way valve 33 as described above. The sensor 31 according to the second embodiment detects the presence or absence of the detection target 9 at a particular position on the path along which the detection target 9 is caused to move by the placing mechanism 8, for example. The particular position is located opposite, in the direction in which the detection target 9 moves, from the position where the detection target 9 faces any of the inlet ports 7. This allows the timing at which the detection target 9 is placed at the position where the detection target 9 faces any one of the inlet ports 7 to be checked based on the result of sensing of the detection target 9 by the sensor 31.

The control unit 32 receives the result of sensing by the sensor 31 and controls the three-way valves 33 in accordance with the result of sensing.

Specifically, first, the control unit 32 controls the respective three-way valves 33 such that the three-way valve 33 of each of the plurality of detection units 2 is in the cleaning state. That is to say, the switching mechanism 3 turns each of the plurality of detection units 2 into the second state.

Next, after the sensor 31 has sensed the detection target 9 at a point in time, the control unit 32 will switch the three-way valve 33 in every one of the plurality of detection units 2 to the detecting state when a certain amount of time passes since the point in time. After that, when another certain amount of time passes since then, the control unit 32 switches the three-way valve 33 to the cleaning state. That is to say, the switching mechanism 3 switches the operating state of every one of the plurality of detection units 2 from the second state to the first state when the certain amount of time passes since the sensor 31 has sensed the detection target 9 and then switches the operating state of the detection unit 2 from the first state to the second state when the certain amount of time further passes after that. This timing to switch the operating state is set such that the detection target 9 sensed by the sensor 31 during the interval between a point in time when the operating state of the detection unit 2 has been switched to the first state and a point in time when its operating state is switched to the second state is placed by the placing mechanism 8 at the position where the detection target 9 faces a particular inlet port 7 (e.g., the inlet port 7, located closest to the detection target 9 in the direction in which the detection target 9 is moving, out of the plurality of inlet ports 7).

The switching mechanism 3 repeats this operation every time the sensor 31 has detected as many detection targets 9 as the detection units 2 (i.e., has detected five detection targets 9). This allows the switching mechanism 3 to switch, every time the sensor 31 has detected as many detection targets 9 as the detection units 2, the operating states of the plurality of detection units 2 at a time. In this manner, the switching mechanism 3 makes the plurality of detection units 2 sequentially perform a series of operating state switches such that their operating state switches from the second state to the first state and then switches to the second state again.

Also, in the second state, the control unit 32 heats the gas sensor 20 for a certain amount of time by causing an electric current to flow through the electro-thermal element 310 as in the first embodiment described above. As the temperature of the gas sensor 20 increases due to heating, desorption of any substance adsorbed to the sensor element Ax is accelerated.

Next, it will be described how the detection system 1 according to the second embodiment operates.

According to the second embodiment, when the detection system 1 starts operating, the pump 11 and the placing mechanism 8 are activated. The placing mechanism 8 carries the plurality of detection targets 9 such that the plurality of detection targets 9 are arranged in line at intervals. The interval between each pair of detection targets 9 is defined to agree with the interval between the inlet ports 7. When the detection system 1 starts operating, the operating state of each of the plurality of detection units 2 is the second state. In this situation, the switching mechanism 3 switches the respective operating states of the plurality of detection units 2 at a time and the processing unit 500 processes the result of detection provided by the detector 4 in each of the plurality of detection units 2. This allows the processing unit 500 to acquire the result of detection based on the substance vaporizing from the detection target 9 or to make a decision, for example, based on the result of detection.

Each of the plurality of detection units 2 operates in the same way as in the first embodiment described above (refer to FIG. 4). Thus, in the second embodiment, the detection unit 2 cannot detect the substance in the second state, either. That is to say, there arises, in the second state, a period in which the detection unit 2 cannot detect any substance.

In contrast, according to the first embodiment, the detection system 1 operates as described above to have had the operating states of the plurality of detection units 2 all switched to the first state at a point in time when as many detection targets 9 as the detection units 2 are placed at respective positions where the detection targets 9 face their corresponding inlet ports 7. This allows the substances vaporizing from the plurality of detection units 2 to be detected at a time.

Subsequently, the switching mechanism 3 performs the operation as described above to switch the operating states of the plurality of detection units 2 to the second state all at a time, thereby cleaning the detectors 4 simultaneously. The placing mechanism 8 moves the plurality of detection targets 9 relatively with respect to the inlet ports 7, thereby placing, following one set of detection targets 9 that are as many as the detection units 2, another set of detection targets 9 that are also as many as the detection units 2 at their respective positions where the detection targets 9 face the plurality of inlet ports 7. By this point in time, the operating states of the plurality of detection units 2 have all been switched to the first state. This allows the substances vaporizing from the plurality of detection units 2 to be detected simultaneously.

This series of operations are performed repeatedly.

Thus, the second embodiment allows the plurality of detection units 2 to detect the substances at a time. Thus, even though there arises, at the same time, a period during which none of the plurality of detection units 2 can detect the substance while the substances vaporizing from the plurality of detection targets 9 are detected, the detection may be made as described above by using the plurality of detection units 2, thus allowing the efficiency of detection to be increased.

The result of detection provided by the detector 4 may also be acquired by the processing unit 500 as in the first embodiment described above. Alternatively, a decision may also be made based on the result of detection, for example.

Next, variations of the present disclosure will be described.

In the first and second embodiments described above, in the first state, the detected gas is allowed to enter the detector 4 through the detecting path 5 but is prohibited from entering the detector 4 through the cleaning path 6. Alternatively, in the first state, as long as the detected gas is allowed to enter the detector 4 through the detecting path 5, the cleaning gas may also be allowed to enter the detector 4. That is to say, the detecting state of the three-way valve 33 may be a state where not only the end of the detecting path 5 but also the end of the cleaning path 6 communicate with the beginning of the introductory path 14.

In the first and second embodiments described above, the switching mechanism 3 includes the three-way valves 33 and switches the operating state of each of the detection units 2 to either the first state or the second state by switching the state of a corresponding one of the three-way valves 33 to either the detecting state or the cleaning state. However, this is only an exemplary implementation of the switching mechanism 3 and should not be construed as limiting. Alternatively, the switching mechanism 3 may include, instead of the three-way valves 33, an on-off valve that selectively allows the detected gas to flow through the detecting path 5 and an on-off valve that selectively allows the cleaning gas to flow through the cleaning path 6 to switch the operating state to either the first state or the second state by opening and closing these on-off valves.

Although the operating states of the detection unit 2 include the first state and the second state in the embodiments described above, the operating states of the detection unit 2 may further include other states. For example, the operating states of the detection unit 2 may further include a third state where the detected gas is prohibited from entering the detector 4 through the detecting path 5 or through the cleaning path 6. In that case, when the operating state switches from the first state to the second state, for example, the operating state may switch from the first state to the third state first and then switch to the second state. Alternatively, when the operating state switches from the second state to the first state, for example, the operating state may switch from the second state to the third state first and then switch to the first state. Optionally, the operating states may further include at least one state other than the first, second, and third states.

In the first and second embodiments described above, the switching mechanism 3 includes the sensor 31 and switching of the operating state of the detection unit 2 is synchronized, based on the result of sensing by the sensor 31, with the placing mechanism's 8 placement of the detection target 9. However, the switching mechanism 3 does not have to include the sensor 31. Alternatively, the switching mechanism 3 may also synchronize switching of the operating state with the placing mechanism's 8 placement of the detection target 9 by any means other than using the sensor 31. For example, switching of the operating state may also be synchronized with the placing mechanism's 8 placement of the detection targets 9 by switching the operating state of the detection unit 2 at regular time intervals without detecting the positions of the plurality of detection targets 9 while causing the detection targets 9 to move at a constant velocity.

In the first and second embodiments described above, the placing mechanism 8 includes a conveyor belt for carrying the detection targets 9. However, this is only an exemplary configuration for the placing mechanism 8. Rather, the placing mechanism 8 may have any other suitable configuration as long as the placing mechanism 8 allows the plurality of detection targets 9 to move relatively with respect to the inlet port(s) 7. Alternatively, the placing mechanism 8 may also move the detection targets 9 relatively with respect to the inlet port(s) 7 by displacing the inlet port(s) 7 without displacing the detection targets 9. Furthermore, the placing mechanism 8 does not have to have such a structure for causing the plurality of detection targets 9 to move only in one direction with respect to the inlet port(s) 7 but may also have a structure (such as a biaxial stage) which may cause the detection targets 9 to move biaxially with respect to the inlet port(s) 7. In that case, the plurality of detection targets 9 may be arranged in columns and rows to form a matrix pattern, instead of being arranged in line.

The implementation of the detector 4 is not limited to the one adopted in the first and second embodiments. For example, if the detector 4 includes a gas sensor, the implementation of the gas sensor is not limited to the above-described one. Rather, when a substance is adsorbed into, coupled to, trapped in, or caused to interact with, an appropriate gas sensor, for example, the weight of the gas sensor, the electrical characteristics (such as the electrical resistance value or the dielectric constant) thereof, the resonant frequency, or the intensity or magnitude of variation in the quantity of light emitted or the dose of a radiation may be acquired as the result of detection. Optionally, the detector 4 may also be a means for quantifying the substance by measuring the absorbance of the substance in a gas phase.

In the first and second embodiments described above, the detector 4 includes the temperature control element 30 for heating the gas sensor 20 and the gas sensor 20 is heated in the second state by the temperature control element 30. However, this is only an example and should not be construed as limiting. Alternatively, the gas sensor 20 does not have to be heated in the second state, and therefore, the detector 4 does not have to include the temperature control element 30. Even if the gas sensor 20 is not heated in the second state, the detector 4 may also be cleaned as long as the cleaning gas may be supplied in the second state to the detector 4 with no detected gas supplied to the detector 4.

The detection target 9 may be anything without limitation. For example, the detection target 9 may be an industrial product, chemicals, an analyte, a processed food, a fresh food, or a plant. The purpose of detecting a substance vaporizing from the detection target 9 is not limited, either. For example, the substance vaporizing from the detection target 9 may be detected for the purpose of quality control, analysis, diagnosis, or any of various other types of decisions or evaluations.

### Recapitulation

A detection system (1) according to a first aspect includes a plurality of detection units (2) and a switching mechanism (3). Each of the plurality of detection units (2) includes: a detector (4) that detects at least one substance contained in a gas phase; a detecting path (5) that allows a first gas, containing a substance vaporizing from a detection target (9), to flow therethrough; and a cleaning path (6) that allows a second gas, not containing the substance vaporizing from the detection target (9), to flow therethrough. The switching mechanism (3) switches an operating state of each of the plurality of detection units (2) from one of a plurality of states to another. The plurality of states includes: a first state where the first gas is allowed to enter the detector (4) through the detecting path (5); and a second state where the first gas is prohibited from entering the detector (4) through the detecting path (5) but the second gas is allowed to enter the detector (4) through the cleaning path (6).

This aspect may reduce the chances of causing a decline in the accuracy of detection of a substance vaporizing from the detection target (9) even when respective substances vaporizing from a plurality of detection targets (9) are sequentially detected.

In a second aspect, which may be implemented in conjunction with the first aspect, the switching mechanism (3) switches the operating state of each of the plurality of detection units (2) alternately from the first state to the second state, and vice versa.

In a third aspect, which may be implemented in conjunction with the first or second aspect, the detection system (1) further includes a single inlet port (7). In each of the plurality of detection units (2), a beginning of the detecting path (5) thereof communicates with the single inlet port (7).

This aspect allows the substance vaporizing from each detection target (9) to be sent from the inlet port (7) via the detecting path (5) to, and detected by, its corresponding detector (4).

In a fourth aspect, which may be implemented in conjunction with the third aspect, the switching mechanism (3) sequentially switches the respective operating states of the plurality of detection units (2) to the first state one after another by shifting a timing to switch the operating state of one of the plurality of detection units (2) from a timing to switch the operating state of another one of the plurality of detection units (2).

This aspect allows the respective substances vaporizing from the plurality of detection targets (9) to be sequentially detected by their corresponding detectors (4) of the plurality of detection units (2).

In a fifth aspect, which may be implemented in conjunction with the fourth aspect, the detection system (1) further includes a placing mechanism (8) that places, in order, one of the plurality of detection targets (9) after another at a position where the detection target (9) faces the single inlet port (7). The switching mechanism (3) switches the operating state in synch with the placing mechanism's (8) placement of the detection targets (9) in order.

In a sixth aspect, which may be implemented in conjunction with the fifth aspect, the placing mechanism (8) includes a mechanism (3) that sequentially moves the plurality of detection targets (9) along a path that passes through the position where each of the plurality of detection targets (9) faces the single inlet port (7). The switching mechanism (3) includes: a sensor (31) that senses the presence or absence of any of the plurality of detection targets (9) at a particular position on the path; and a control unit (32) that controls, in accordance with a result of sensing by the sensor (31), a timing to switch the operating state.

This aspect allows the switching mechanism (3) to accurately synchronize, in accordance with the result of sensing by the sensor (31), switching of the operating state and placement of the detection targets (9) by the placing mechanism (8) with each other.

In a seventh aspect, which may be implemented in conjunction with the first or second aspect, the detection system (1) further includes a plurality of inlet ports (7). The beginning of the detecting path (5) of each of the plurality of detection units (2) communicates with a corresponding one of the plurality of inlet ports (7).

This aspect allows the substance vaporizing from each of the plurality of detection targets (9) to be sent from a corresponding one of the plurality of inlet ports (7) through a corresponding one of the detecting paths (5) to, and detected by, a corresponding one of the detectors (4), thus contributing to increasing the efficiency of detection.

In an eighth aspect, which may be implemented in conjunction with the seventh aspect, the switching mechanism (3) switches the respective operating states of the plurality of detection units (2) to the first state simultaneously.

This aspect allows the respective substances vaporizing from the plurality of detection targets (9) to be detected simultaneously by the plurality of detection units (2).

In a ninth aspect, which may be implemented in conjunction with the eighth aspect, the detection system (1) further includes a placing mechanism (8) that sequentially places the plurality of detection targets (9) one by one at a plurality of positions, at each of which one of the plurality of detection targets (9) faces a corresponding one of the plurality of inlet ports (7). The switching mechanism (3) switches the operating state in synch with the placing mechanism's (8) sequential placement of the detection targets (9).

In a tenth aspect, which may be implemented in conjunction with the ninth aspect, the placing mechanism (8) includes a mechanism that sequentially moves the plurality of detection targets (9) along a path that sequentially passes through the positions where the plurality of detection targets (9) face one of the plurality of inlet ports (7) after another (7). The switching mechanism (3) includes: a sensor (31) that senses the presence or absence of any of the plurality of detection targets (9) at a particular position on the path; and a control unit (32) that controls, in accordance with a result of sensing by the sensor (31), a timing to switch the operating state.

This aspect allows the switching mechanism (3) to accurately synchronize, in accordance with the result of sensing by the sensor (31), switching of the operating state and placement of the detection targets (9) by the placing mechanism (8) with each other.

In an eleventh aspect, which may be implemented in conjunction with any one of the first to tenth aspects, the detector (4) includes a sensor element (Ax) having an electrical resistance value that changes when adsorbing a substance.

In a twelfth aspect, which may be implemented in conjunction with any one of the first to eleventh aspects, the detector (4) includes a sensor array including a plurality of sensor elements (Ax) having mutually different sensitivities.

A detection method according to a thirteenth aspect is a method for detecting, using a detection system (1), a substance vaporizing from a detection target (9). The detection system (1) includes a plurality of detection units (2). Each of the plurality of detection units (2) includes: a detector (4) that detects at least one substance contained in a gas phase; a detecting path (5) that allows a first gas, containing a substance vaporizing from a detection target (9), to flow therethrough; and a cleaning path (6) that allows a second gas, not containing the substance vaporizing from the detection target (9), to flow therethrough. The detection method includes switching an operating state of each of the plurality of detection units (2) from one of a plurality of states to another. The plurality of states includes: a first state where the first gas is allowed to enter the detector (4) through the detecting path (5); and a second state where the first gas is prohibited from entering the detector (4) through the detecting path (5) but the second gas is allowed to enter the detector (4) through the cleaning path (6).

This aspect may reduce, in detecting the substance vaporizing from the detection target (9), the chances of causing a decline in detection accuracy even when sequentially detecting respective substances vaporizing from a plurality of detection targets (9).

### REFERENCE SIGNS LIST

1 Detection System
2 Detection Unit
3 Switching Mechanism
31 Sensor
32 Control Unit
4 Detector
5 Detecting Path
6 Cleaning Path
8 Placing Mechanism
9 Detection Target
Ax Sensor Element

1. A detection system comprising a plurality of detection units and a switching mechanism,
each of the plurality of detection units including:
a detector configured to detect at least one substance contained in a gas phase;
a detecting path configured to allow a first gas, containing a substance vaporizing from a detection target, to flow therethrough; and
a cleaning path configured to allow a second gas, not containing the substance vaporizing from the detection target, to flow therethrough,
the switching mechanism being configured to switch an operating state of each of the plurality of detection units from one of a plurality of states to another, and
the plurality of states including: a first state where the first gas is allowed to enter the detector through the detecting path; and a second state where the first gas is prohibited from entering the detector through the detecting path, but the second gas is allowed to enter the detector through the cleaning path.

2. The detection system of claim 1, wherein
the switching mechanism is configured to switch the operating state of each of the plurality of detection units alternately from the first state to the second state, and vice versa.

3. The detection system of claim 1, further comprising a single inlet port, wherein
in each of the plurality of detection units, a beginning of the detecting path communicates with the single inlet port.

4. The detection system of claim 3, wherein
the switching mechanism is configured to sequentially switch the respective operating states of the plurality of detection units to the first state one after another by shifting a timing to switch the operating state of one of the plurality of detection units from a timing to switch the operating state of another one of the plurality of detection units.

5. The detection system of claim 4, further comprising a placing mechanism configured to place, in order, one of the plurality of detection targets after another at a position where the detection target faces the single inlet port, wherein
the switching mechanism is configured to switch the operating state in synch with the placing mechanism's placement of the detection targets in order.

6. The detection system of claim 5, wherein
the placing mechanism includes a mechanism configured to sequentially move the plurality of detection targets along a path that passes through the position where each of the plurality of detection targets faces the single inlet port, and
the switching mechanism includes: a sensor configured to sense presence or absence of any of the plurality of detection targets at a particular position on the path; and a control unit configured to control, in accordance with a result of sensing by the sensor, a timing to switch the operating state.

7. The detection system of claim 1, further comprising a plurality of inlet ports, wherein
a beginning of the detecting path of each of the plurality of detection units communicates with a corresponding one of the plurality of inlet ports.

8. The detection system of claim 7, wherein
the switching mechanism is configured to switch the respective operating states of the plurality of detection units to the first state simultaneously.

9. The detection system of claim 8, further comprising a placing mechanism configured to sequentially place the plurality of detection targets one by one at a plurality of positions, at each of which one of the plurality of detection targets faces a corresponding one of the plurality of inlet ports, wherein
the switching mechanism is configured to switch the operating state in synch with the placing mechanism's sequential placement of the detection targets.

10. The detection system of claim 9, wherein
the placing mechanism includes a mechanism configured to sequentially move the plurality of detection targets along a path that sequentially passes through the positions where the plurality of detection targets sequentially face one of the plurality of inlet ports after another, and
the switching mechanism includes: a sensor configured to sense presence or absence of any of the plurality of detection targets at a particular position on the path; and a control unit configured to control, in accordance with a result of sensing by the sensor, a timing to switch the operating state.

11. The detection system of claim 1, wherein
the detector includes a sensor element having an electrical resistance value that changes when adsorbing a substance.

12. The detection system of claim 1, wherein
the detector includes a sensor array including a plurality of sensor elements having mutually different sensitivities.

13. A detection method for detecting, using a detection system, a substance vaporizing from a detection target,
the detection system comprising a plurality of detection units, each of the plurality of detection units including:
a detector configured to detect at least one substance contained in a gas phase;
a detecting path configured to allow a first gas, containing a substance vaporizing from a detection target, to flow therethrough; and
a cleaning path configured to allow a second gas, not containing the substance vaporizing from the detection target, to flow therethrough,
the detection method comprising switching an operating state of each of the plurality of detection units from one of a plurality of states to another, and
the plurality of states including: a first state where the first gas is allowed to enter the detector through the detecting path; and a second state where the first gas is prohibited from entering the detector through the detecting path, but the second gas is allowed to enter the detector through the cleaning path.
